(19) United States
(12) Patent Application Publication
(10) Pub. No.: US 2013/0004472 A1
(43) Pub. Date: Jan. 3, 2013

(54) MEANS AND METHODS FOR IMPROVING THE DEVELOPMENT AND MATURATION OF EGGS AND/OR SPERM IN FISH USING HORMONES PRODUCED BY TRANSPLANTED CELLS

(76) Inventors: Herman Pieter Spaink, Oegstgeest (NL); Guido Everard Elisabeth Johannes Maria Van Den Thillart, Leiderdorp (NL); Denhi Schnabel Peraza, Leiden (NL)

(21) Appl. No.: 13/597,069

(22) Filed: Aug. 28, 2012

Related U.S. Application Data
(62) Division of application No. 11/883,200, filed on Oct. 16, 2007.

(30) Foreign Application Priority Data
Jan. 26, 2005 (EP) 05075204.7
Feb. 28, 2005 (EP) 05075490.2

(51) Int. Cl. A61K 35/12; C12N 5/10; A61P 5/06; C12N 5/07

(52) U.S. Cl. 424/93.21; 424/93.7; 435/325

(57) ABSTRACT

Methods and/or hormone-producing cells for improving the development and/or maturation of eggs and/or sperm in fish using hormone administration. The methods may comprise providing the fish with cells producing the hormone. Preferred hormones are fertility hormones such as luteinizing hormone (LH), follicle-stimulating hormone (FSH) or chorionic gonadotropin (CG) or a functional part, derivative and/or analogue thereof.

### CROSS-REFERENCE TO RELATED APPLICATIONS

This application is a divisional of co-pending U.S. patent application Ser. No. 11/883,200, filed Oct. 16, 2007, which is a national phase entry under 35 U.S.C. §371 of International Patent Application PCT/NL2006/000043, filed Jan. 26, 2006 designating the United States, published in English as International Patent Publication W02006/080841A1 on Aug. 3, 2006, which application claims the benefit under Article 8 of the Patent Cooperation Treaty to European Patent Applications Serial No. 05075204.7 filed Jan. 26, 2005 and Serial No. 05075490.2 filed Feb. 28, 2005; the disclosures of the entirety of each of which are hereby incorporated herein by this reference.

### TECHNICAL FIELD

The disclosure relates to the field of fish culture. In particular, it relates to the field of hormone-driven improvement of the development and maturation of eggs and/or sperm in fish.

### BACKGROUND

Many fish species mature in response to environmental factors. These factors, such as light cycle, temperature, season, pressure, and energy reserves, are sensed by the animal and control the inhibitory action of hypothalamic centers on the pituitary. In this way, the production of gonadotropins by the pituitary is normally depressed and activated only under certain environmental conditions. When activated, the pituitary releases gonadotropins that stimulate the growth and development of both male and female gonads.

For fisheries, it is important that maturation can typically be stimulated artificially by regular injections with the same hormones, which often consist of crude pituitary extracts. The regular injections overrule to some extent the environmental triggers for the development of both the male and female gonads. However, the current practice of artificially improving the development and/or maturation of eggs and/or sperm using hormones is not completely satisfactory. For instance, in eels, which are still immature when they commence their spawning migration, the females have to be treated weekly during 3-5 months before oocytes are ripe enough for ovulation. This is a time-consuming procedure and stressful for the fish.

### DISCLOSURE OF THE INVENTION

Provided is a method for improving the development and/or maturation of eggs and/or sperm in fish using hormone administration comprising providing the fish with cells producing the hormone. The cells that are transplanted into the fish release one or more hormones, thereby at least reducing the need for regular injections with the hormone(s) themselves. The cells may be transplanted in various ways as long as the secreted hormone(s) are released into the circulation system and reach all tissues and, in particular, the sexual organs, in sufficient quantity. The cells may be transplanted anywhere in the body. Implants, consisting of hormone(s)-producing cells, are typically used to bypass the pituitary gland in order to produce hormones, such as luteinizing hormone (LH), follicle-stimulating hormone (FSH), or chorionic gonadotropin (CG). The implants with hormone-producing cells are preferably inserted into sites that have access to the bloodstream. Preferred methods of insertion are intra-peritoneal and subcutaneous injection. Sites with access to the bloodstream are very well suited for cells that produce secondary hormones such as LH, FSH, and CG.

Transplantation of cells is often used in mammals and a lot of experience has been obtained with respect to methods to transplant and maintain cells for at least some time. Considering that the fish are typically killed after spawning or harvesting of the ripened eggs or sperm, there is no great need for control over the transplanted cells. Important is that the cells remain present in sufficient numbers to allow complete development and maturation of the gonads. Thus, the number of cells may not be less than required for development and maturation and the cells may not be (or become) so numerous that they impede the development and maturation of the gonads or the general health of the animal.

The level of production of the hormone by the cells is not very critical. The number of injected cells will depend on the total quantity required for the maturation. The hormone production will be quantified by a bio assay. The hormone release of the injected cells needs to be sufficiently high to stimulate the development and/or maturation of eggs and/or sperm. The upper boundary for expression of the hormone is not critical as over-expression of a fertility hormone is not toxic in itself and does not negatively affect the stimulation of maturation and/or development of eggs and/or sperm.

In the mammalian world, several types of grafting aids have been developed to allow for prolonged stay of the cells or to allow differentiation of the cells. These aids can, of course, also be used herein. Such aids include, but are not limited to, collagen or synthetic matrices for the grafting and attachment of cells.

As the cells in many cases need not be present for a very long time, it is possible to transplant fish cells from many different species into a recipient fish. If the evolutionary difference between the transplanted cells and the recipient is large, it is likely that the recipient fish will mount an immune response to the transplanted cells (the graft). However, as the cells often need only be present for a limited amount of time, such immune response can typically be tolerated. To increase the robustness and predictability of the procedure, it is preferred that the graft is derived from the same genus or family as the recipient fish species. Preferably, the two are from the same species. As fish are typically outbred populations, there are immunological differences between fish of the same species. This is typically not a problem as shown in one of our examples of experiments with eels, however, it is possible to further match the graft and the recipient for common immunological markers. Typical markers are major and minor histocompatibility antigens. The grafting of the transplanted cells may further be facilitated by providing the recipient fish with immunosuppressants such as cyclosporin.

The transplantation of hormone-producing cells hereof can be used for improving the maturation of eggs and/or sperm, the fertility of the eggs and/or sperm, the insemination of eggs, the quality of the resulting embryos, the survival of fertilized and unfertilized eggs and the survival of embryos. These improvements all lead to improved development and maturation of eggs and/or sperm in fish. The term “development and maturation of eggs and/or sperm in fish” is, therefore, not limited to the natural process of spawning but also relates to artificial methods for egg insemination. Thus, it also relates to the harvesting of unfertilized eggs and/or sperm from fish treated with a method hereof. The unfertilized eggs may also be used for other purposes than the creation of progeny. A non-limiting example thereof is the production of eggs for human consumption such as caviar.

The development and maturation of eggs and/or sperm in fish can be stimulated in various ways. In one embodiment, the development and maturation of eggs and/or sperm in fish is said to be stimulated when the absolute number or the quality of the eggs, sperm or embryos resulting from fertilized eggs is increased.

Reproduction is a highly regulated biological process. Different aspects of reproduction are regulated by different hormones. However, several hormones can produce more or less similar effects when expressed by a cell that is transplanted into a fish. These hormones include: Growth hormone, Corticoliberin (Adreno Corticotrope hormone), Thyroid-stimulating hormone, FSH, LH, Prolactin, CG (Chorionic gonadotropin), MG (Menopause gonadotropin), Somatotropin, or a combination thereof. The hormones mentioned above are also known under different names. As the underlying amino acid sequence is the same, the hormones referred to by the synonyms are also within the scope hereof. For instance, Growth hormone is sometimes also referred to as Somatotropin, somatotropic hormone, hypophysis growth hormone, somatotropic hormone or STH. Corticoliberin is also referred to as releasing corticotropin hormone. Adreno Corticotrope hormone is also referred to as Corticotropin, adrenocorticotropin, adrenotropin, corticotropin, ACTH or adrenocorticotropic hormone. Thyroid-stimulating hormone is also referred to as TSH, thyrotropin or thyrotropic hormone. FSH is also referred to as Follicle-stimulating hormone, follitropin or gametocinetic hormone. LH is also referred to as Luteinizing hormone, Luteotropin or interstitial cell stimulating hormone (ICSH). Prolactin is also referred to as PRL, lactogenic hormone, mammotropic hormone, galactopoietic hormone or lactotropin. CG (Chorionic gonadotropin) is also referred to as Chorionic gonadotropic hormone, choriogonadotropin or chorionic gonadotropin and Menopause gonadotropin (MG) is also referred to as urogonadotropin, menotropin or Menopause gonadotropic hormone.

In a preferred embodiment, the hormone is a hormone directly involved in the development and maturation of eggs. Such fertility hormones are typically produced by the pituitary or the sexual organs. In a preferred embodiment, the fertility hormone comprises luteinizing hormone (LH), follicle-stimulating hormone (FSH), or chorionic gonadotropin (CG) or a functional part, derivative and/or analogue of such a hormone. These hormones are very potent stimulators of the development and maturation of eggs and/or sperm in fish. These hormones are very conserved in nature and hardly have a species barrier. For instance, the presence of human fertility holt cones in urine can be detected by incubating them with frog eggs. Similarly, human chorionic gonadotropin (hCG) also works on eel and carp and salmon pituitary extracts work on many different fish species, such as eel, seabream and trout.

It is possible that the recipient develops an immune response against a heterologous hormone. Although this immune response is typically too late to affect the stimulation of maturation and/or development of eggs and/or sperm, it is preferred that the hormone is a fish hormone or a functional part, derivative and/or analogue thereof. This limits the divergence between the provided and the endogenous hormone, thereby at least in part limiting the development of an immune response against the provided hormone in the recipient. Preferably, the hormone is derived from a species that belongs to the same genus as the recipient. In this way, the chance that an immune response is developed is further reduced. In a particularly preferred embodiment, a provided hormone is immunologically identical to the equivalent thereof in the recipient. This completely prevents the development of any detrimental immune response against the provided hormone.

The cells can either express the desired hormone without manipulation or can be manipulated to express the desired hormone. When the cells do not express the hormone already or do not express sufficient hormone, they can be provided with the genetic information for expressing the hormone.

In a preferred embodiment, the cells are provided with the genetic infatuation to express the hormone. This can be done by providing the cells with expression cassettes comprising coding sequences for the hormones. However, it is also possible to activate the endogenous genes by inserting an active regulatory sequence near the coding sequence(s) for the respective hormones. This can be done, for instance, through homologous recombination.

The LH and FSH proteins belong to a family of related proteins. Both share the characteristic that they are functional as heterodimers consisting of a common α-subunit and a different β-subunit. In the case of hormones that consist of more than one protein chain, it is possible that cells do not express all of the chains needed to generate the hormone. In these cases, only expression cassettes are required for the chain(s) that are lacking. Thus, if one or more but not all of the subunits of the hormone are adequately expressed in the cells, one only needs to express the remaining subunit(s) in the cell. If none of the subunits are expressed, one has to manipulate the cells such that all of the subunits are expressed at adequate levels. In a preferred embodiment, the cells are provided with expression cassettes for the subunits of the hormone. In a preferred embodiment, the cells are provided with expression cassettes for the three protein chains that make up LH and FSH (i.e., for the common α-subunit and the unique β-subunits for each of the hormones), or in separate cell lines the combination of βLH+α and βFSH+α are expressed. Thus, in a preferred embodiment, the cells are genetically modified to express the hormone(s). Preferably, the cells are provided with one or more genes encoding the hormone(s).

The cells can be primary cells or cell lines that are cultured in vitro for an extended period. In a preferred embodiment, the cells are derived from a clonal population of cells. In this way, the cells can be subjected to detailed quality control prior to use. This also allows for the generation of cell banks that have the same property. Moreover, a clonal population can be subjected to further manipulations. For instance, if one wants to reduce immune responses in the recipient, it is possible to knock out expression of major and/or minor histocompatibility antigens. Thus, in a preferred embodiment, the cells have been selected for a reduced immunogenicity in the recipient.

A cell for use in a method or use herein can be a primary cell or a cultured cell. Preferably, the cell is a cultured cell, more preferably an immortalized cultured cell. Cultured cells are typically cell lines. Cell lines can be propagated for at least five passages without substantial change in the phenotype of the cell. Immortalized cell lines can be passaged at least 50 times without undergoing such phenotypic change. Immortalized cells can be obtained from primary cells in various ways. Preferably, an immortalized cell is obtained from a culture of primary cells that has undergone the crisis that is typically for primary cells in culture. In another preferred embodiment, the cell has become immortalized through introduction of one or more genes into a primary cell.

Methods hereof may be used for all types of fish. Preferred fish are eel, seabass, seabream, halibut, salmon, trout, cod, carp, catfish, and sturgeon. However, the methods are particularly suited for diadromous and preferably semelparous fish. These fish take a long time before spawning and typically do not all respond similarly to outside signals. With a method hereof, it is possible to stimulate the development and maturation of the fish at least in part independently of the environmental stimuli. This introduces a large amount of predictability towards the starting point for the fish culture. In diadromous fish, it is further possible to synchronize egg development and maturation such that work can be better scheduled in the production process.

Further provided is an isolated and/or recombinant fish cell that produces a fertility hormone. In a preferred embodiment, the cell is genetically modified to express the hormone. Further provided is a fish cell provided with the capacity to express a fertility hormone. Preferably, the fish cell is provided with a recombinant and/or isolated nucleic acid sequence encoding the fertility hormone. If the hormone consists of one or more subunits, the fish cell is preferably provided with an isolated and/or recombinant nucleic acid sequence encoding at least one subunit of the hormone. Preferably, the fish cell is provided with nucleic acid sequence encoding all subunits of the hormone. Preferably, the cell is a cell of a consumer fish. In a preferred embodiment, the cell originates from eel, seabass, seabream, halibut, salmon, trout, cod, carp, catfish or a sturgeon. Preferably, the cell is a cell as described above, i.e., a primary cell or a cell derived from a cell line that is cultured in vitro for an extended period. In a preferred embodiment, the cells are derived from a clonal population of cells. Preferably, the cell is a cultured cell, more preferably an immortalized cultured cell.

In a preferred embodiment, use is made of implants of cells that can be cultured. Furthermore, these cells are preferably clonal and preferably selectable for characteristics. It is, however, also possible to make use of primary cultures, tissue, fertilized eggs, or embryonic material. A method for making transgenic fish eggs has been published (Morita et al. 2004, Transgenic Research 13:551). However, the mentioned transgenic fish eggs cannot be further propagated. Furthermore, the expression of the introduced gene(s), as described by Morita et al. is not stable. Selection of the introduced genes in eggs is difficult and using fish eggs as biorectors is rather time consuming in relation to culturable cells because micro-injection for each of the eggs has to be used, whereas in cell cultures, we can make use of standard transfection technology. Culturable cells have the advantage that they are easily stored and can be made available at any time. Furthermore, there are no ethical problems with working with culturable cells as compared to genetically modified eggs. Injection of a suspension of cultured cells is rather easy as compared to implantation of eggs and/or embryos.

Further provided is a fish that comprises a cell hereof. Preferably, the fish is a consumer fish. Preferably, the fish is a consumer juvenile or adult fish. The time to reproduction is preferably short. Primary embryonic material has the disadvantage that it might not be available, or not available, in sufficient quantities. In a preferred embodiment, the fish is an eel, seabass, seabream, halibut, salmon, trout, cod, carp, catfish or a sturgeon. In a preferred embodiment, the fish comprises non-genetically modified sperm cells, oocytes and/or progenitors thereof.

### BRIEF DESCRIPTION OF THE FIGURES

FIG. 1: Dendrogram based on alignment of the LHβ (LHβ), FSHβ (FSHβ) originating from α (a) of human (Hs), mice (Mm), rat (Rn), zebrafish (Dr) and eel (Aj).

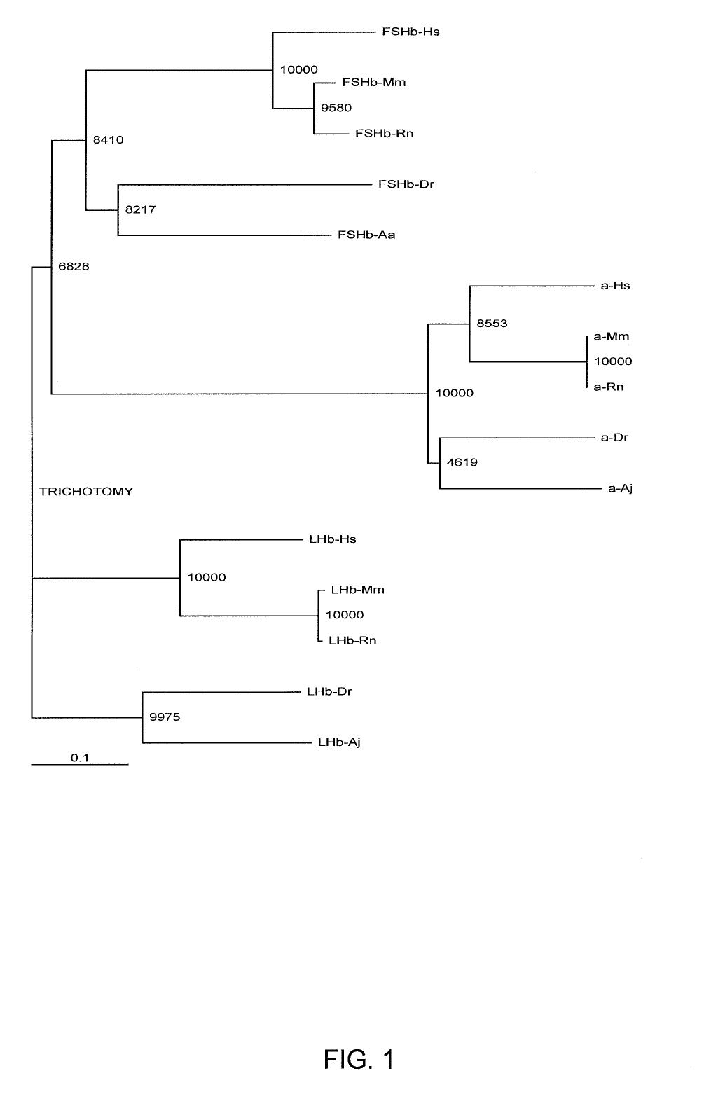

FIG. 2: p3XFLAG-CMV-9 expression vector.

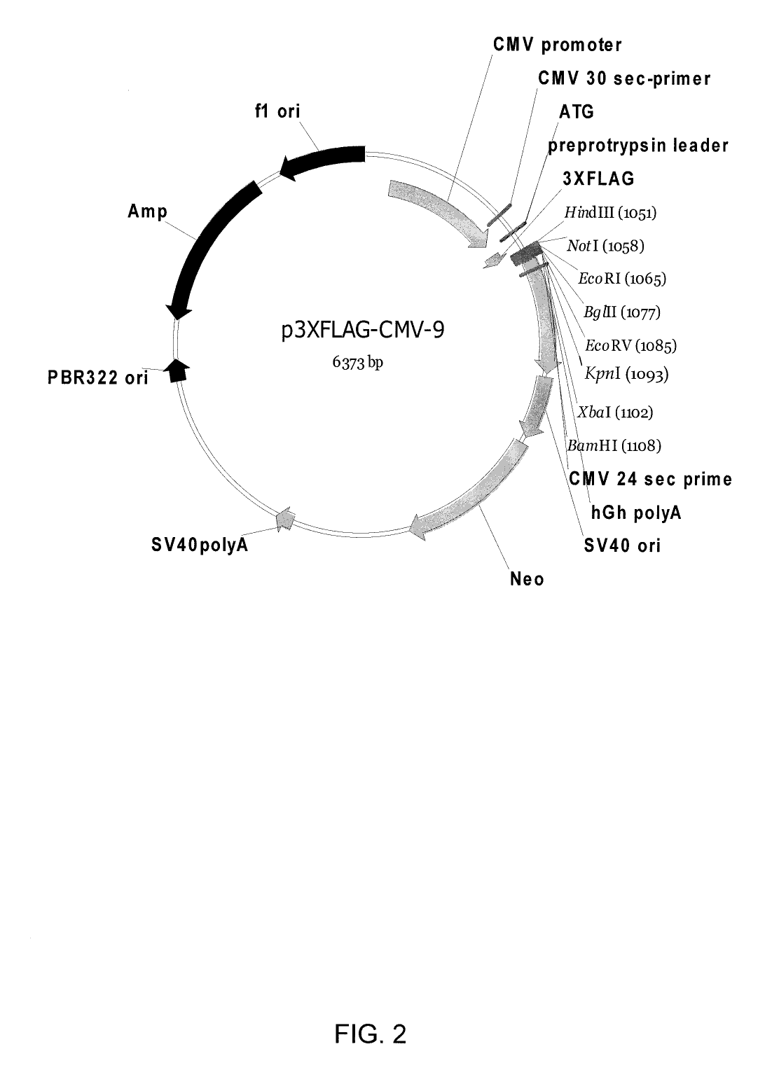

FIG. 3: Cloning strategy for the expression of LHβ.

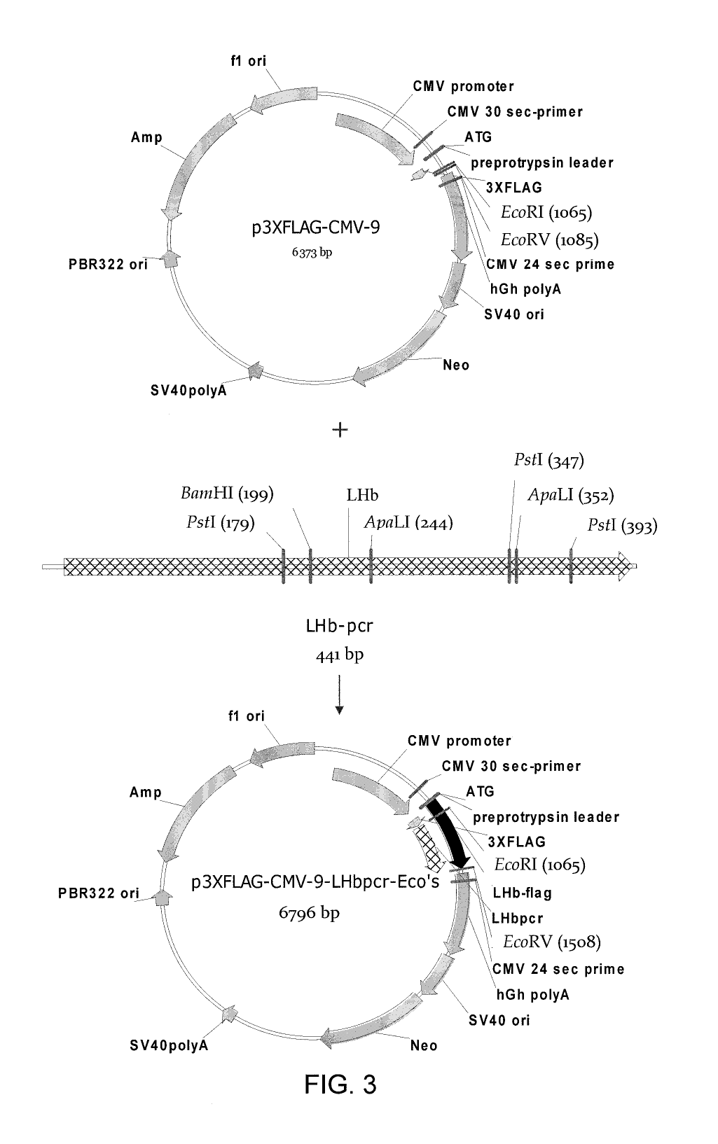

FIG. 4: The predicted amino acid sequence of LHβ gives a protein of 187 amino acids with a molecular weight of 20593.8.

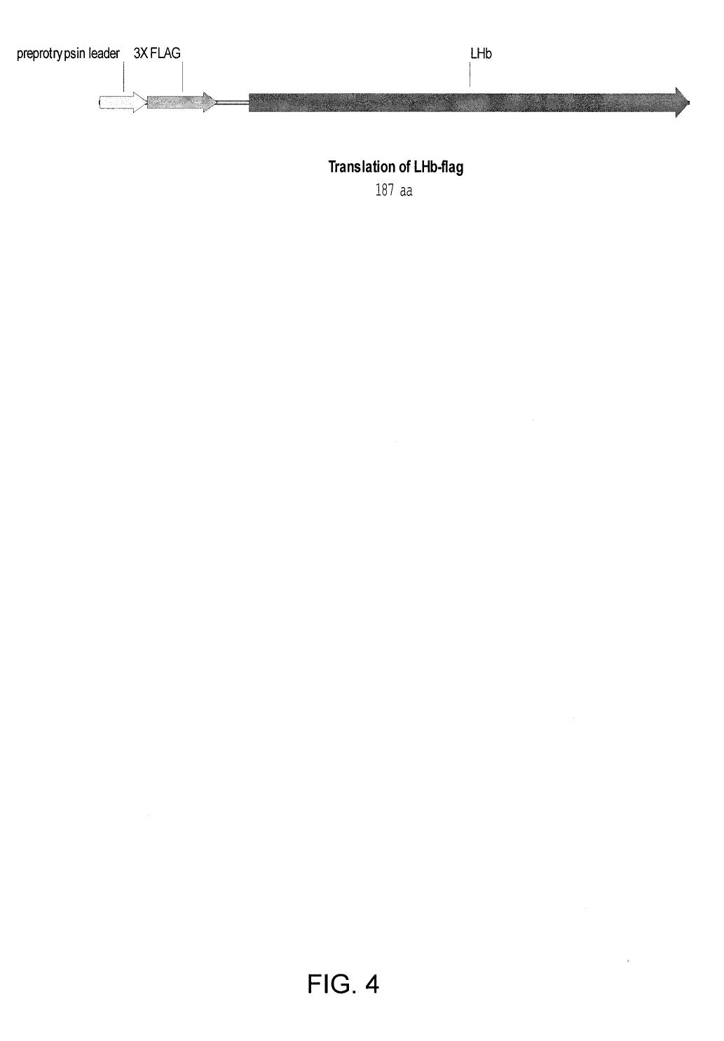

FIG. 5: Cloning strategy for the expression of FSHβ.

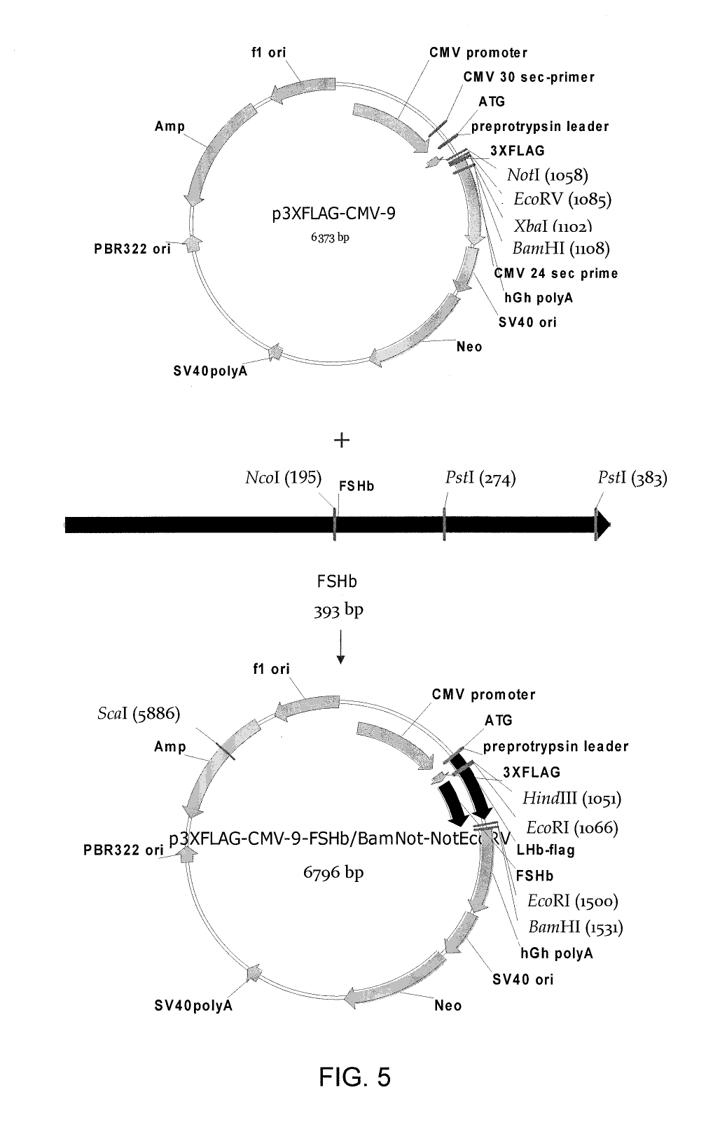

FIG. 6: The predicted amino acid sequence of FSHβblu and FSHβ gives a protein of 174/183 amino acids with a molecular weight of 19062.24/19965.21, respectively, to the two different strategies of cloning.

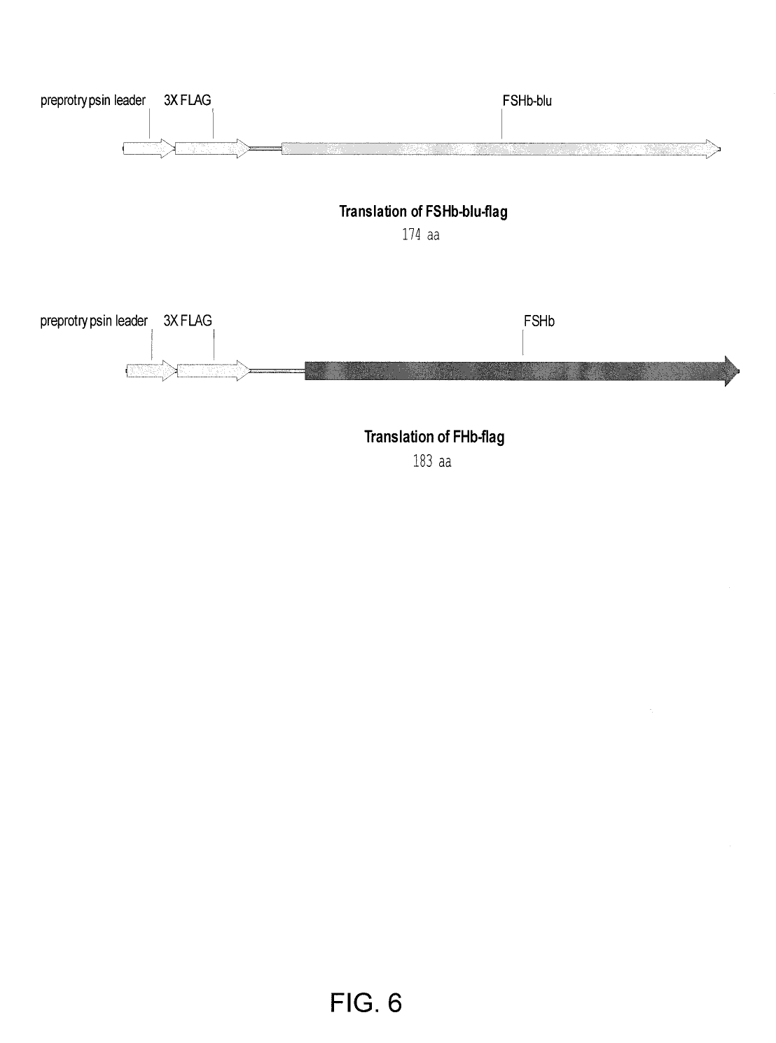

FIG. 7: Cloning strategy for the expression of α.

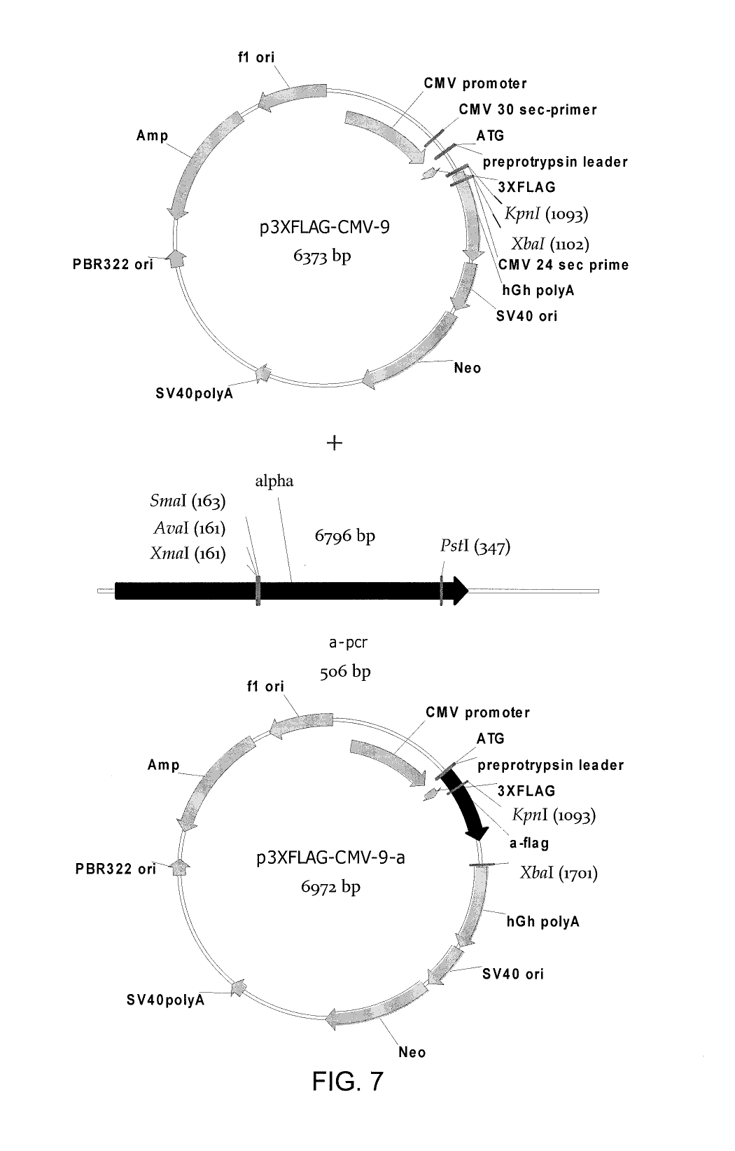

FIG. 8: The predicted amino acid sequence gives a protein of 190 amino acids with a molecular weight of 21083.24.

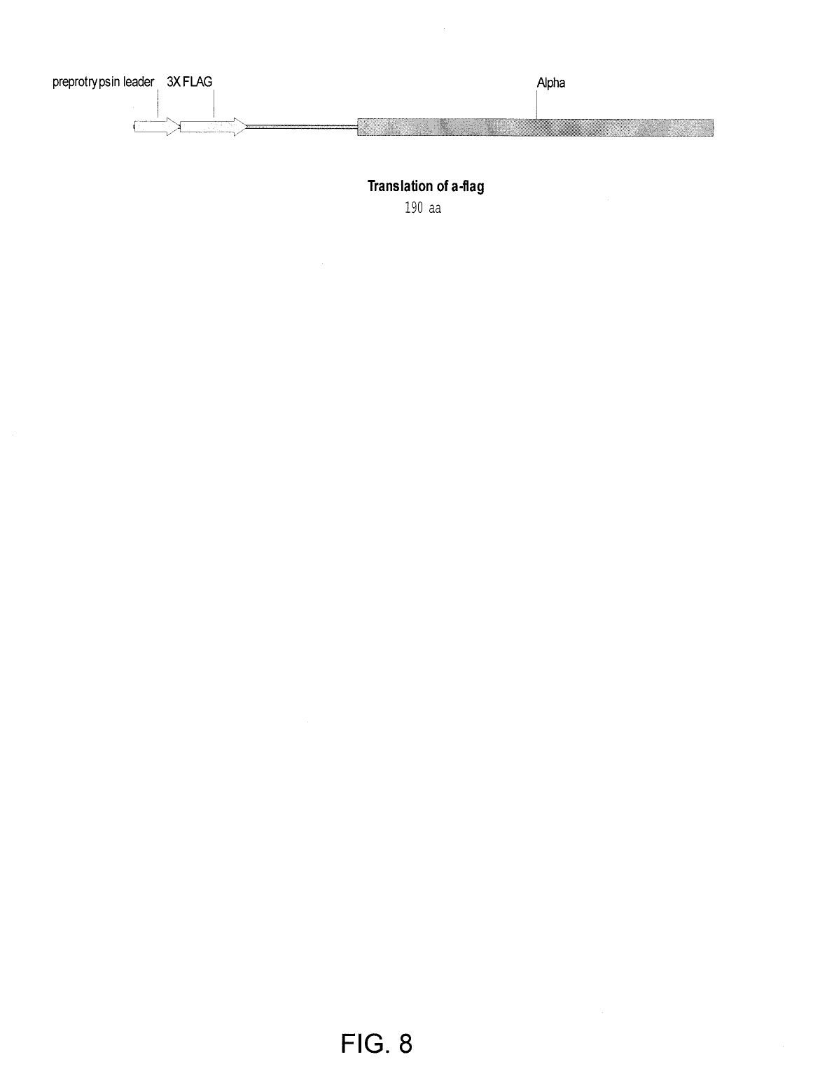

FIG. 9: Transfection of ZF4 cells with pEYFP-N1. Transfection is observed after 24 hours of transfection.

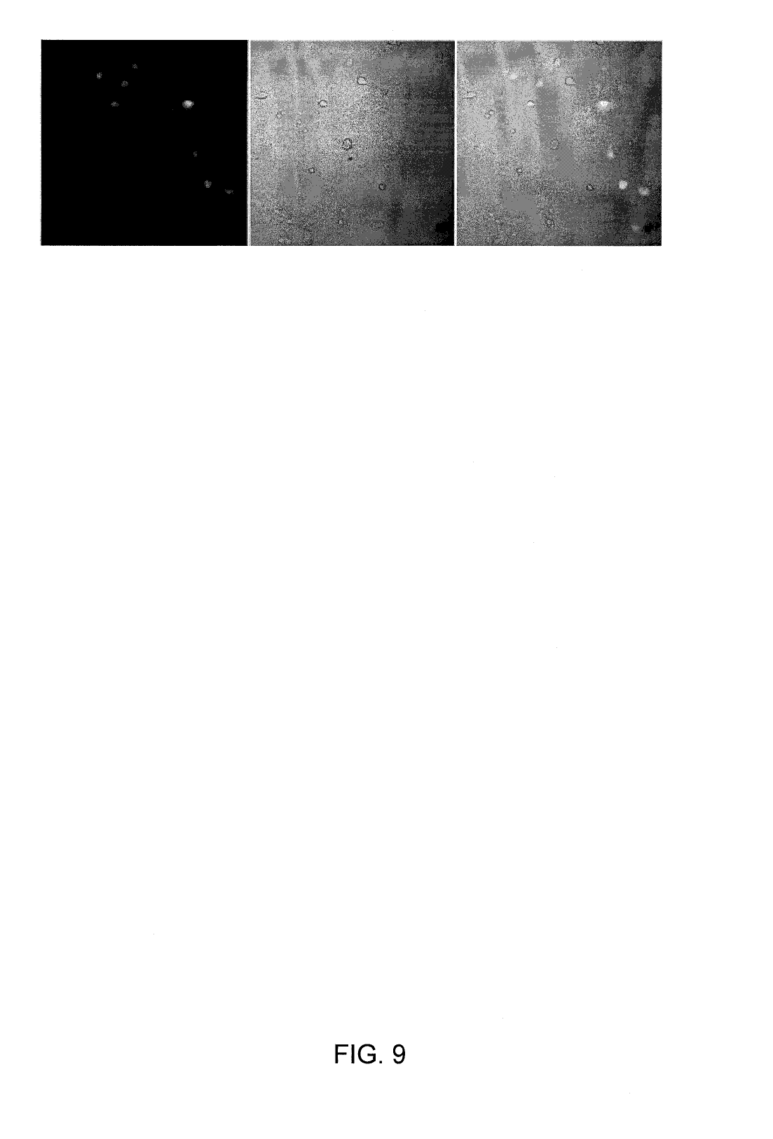

FIG. 10: Immunohistochemistry with anti-FLAG antibody in co-transfected cells with pEYFP-N1.

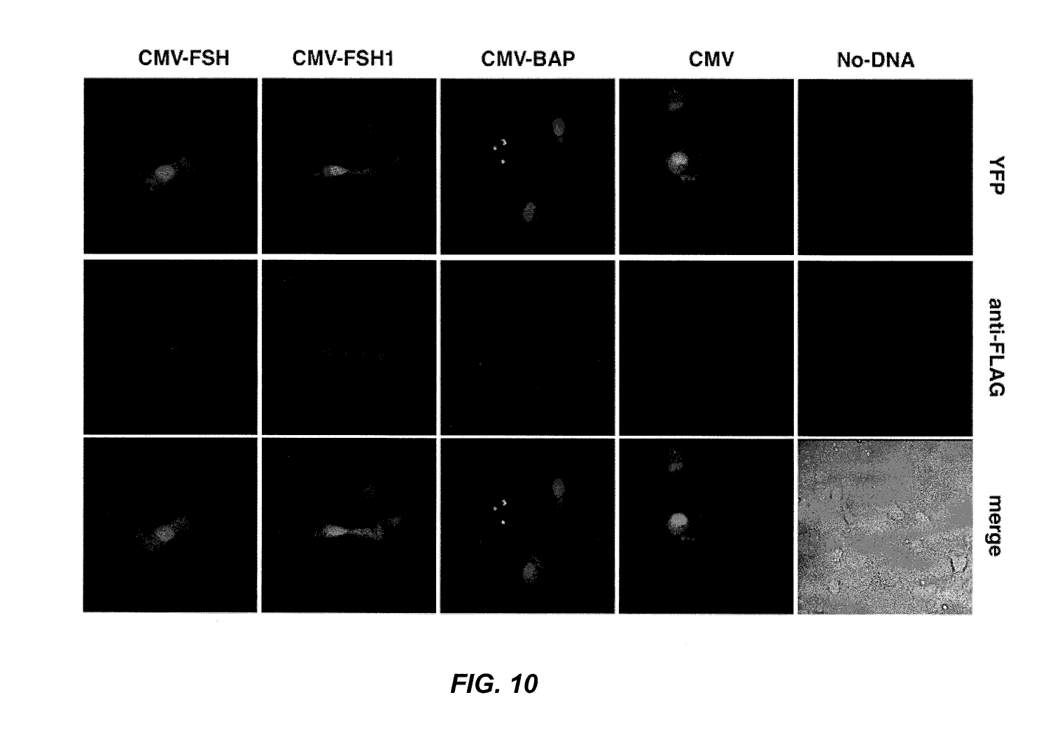

FIG. 11: Western blot with anti-FLAG. The antibody recognized a protein of the expected size for FLAG-BAP.

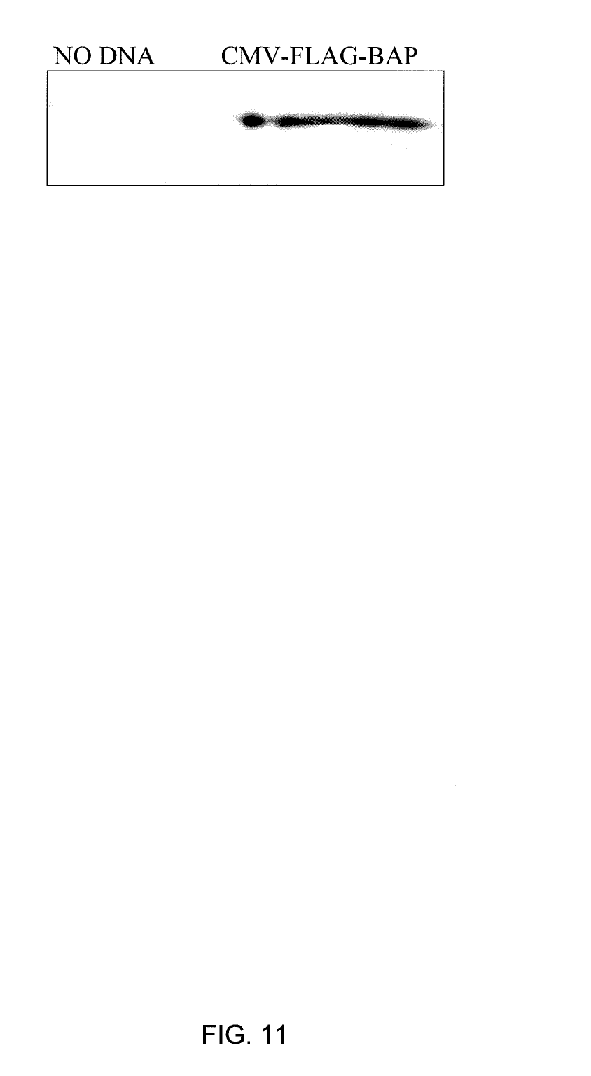

FIG. 12: Staining for β-galactose indicated the occurrence of β-gal-ZF4 cells that were injected subcutaneously in silver eels. From the same eel, skin/muscle tissue was dissected from the injected area and an area farther towards the tail. Subcutaneous fat tissue is easily recognizable in the figures, as is the dermis and the muscle layer.

FIG. 13: Eye Index (EI) changes in regard to the initial stage (%) of silver eels during four weeks (W1-W4) of treatment with hormone-producing cell (HPC) or with carp pituitary extracts (CPE). For the initial stage and each of the weeks six full-grown female silver eels were taken (weight 1300±300 g).

### DETAILED DESCRIPTION OF THE INVENTION

### Examples

### Cloning of LHβ, FSHβ and a Zebrafish Genes

The genes of zebrafish are already published LHβ (AY424304), FSHβ (AY424303) and α (AY424306), also the genes of eel are reported for LHβ (AB175835) in Anguilla japonica; FSHβ (AY169722) in Anguilla anguilla and for α (AB175834) in Anguilla japonica. In order to clone LHβ, FSHβ and α, primers were designed based on the cDNA sequence of each one:

The sequence used for the design of the primers of the LHβ was:

(SEQ ID NO: 1)
atatataaat ctggacacgc agagacactt acaacagcct gctgagcaac cgcaacgcct
gtcaagatgt tattggctgg aaatggtgtc ttctttctct tctctttgtt tttcctgctg
gcggctgctc agagcttggt ttttccacgc tgtgagctag taaatgagac ggtatcggtg
gaaaaagagg gctgtccaaa atgcctggtg tttcagacca ccatctgcag cggccactgc
gtaacaaggg atcccgttta caagagcccg ttttccaccg tccaccagac agtgtgcatg
taccgggacg tccgctatga gaccattaac ctgcccgact gttccgccgg cgtggacccg
cagatcacat acccggtggc gctgagctgc gactgcagtc tgtgcaccat aaacacttcc
gactgcacca tccagagcct gcagcccgac ttctgcatgt cccagagaga ggatttcccc
gcatactaga cctcgggcaa ctcacgtcaa cctacgcaca tagtcgagct cagcattatt
agccctcctg tatgtttttt ccattaatat atatactttc aagacactag tattcagctt
aaagtgacat ttaaagacta aactaggtta attaggggga aaagtagagt aagtcattgt
ataatagtgg tttgttctgg agacaatcca aaactaatat tgcttaaggg ggctaataaa
attgacctta aaatgaattt aaataattta aaaactgcat ttattctagt cgaaataaaa
gaaataagac tttctttaga agaaaaaaca ttataggaaa tactgcaaaa aaattcctga
atctgttcaa catcattcgg gaaatcaaag gagggctaat aactgtgact tcagctgtac
atcaataaag aggctggttc ttaaattcaa aaaaaaaaaa aaaaaaaaaa aaaaaaaa

The sequence used for the design of the primers of the FSHβ was:

(SEQ ID NO: 2)
ggtctccacg aaactcccgc agatgaggat gcgtgtgctt gttctggcgc tgctgttgcc
ggtgttaatg agcgcagaat cagaatgcag gtgcagctgt cgactcacca acatctccat
cactgtggag agcgaagaat gtgggagctg cgtcacaatc gacaccacag cctgtgcagg
actatgctgg acaatggatc gagtttaccc tagttccatg gcacagcaca cccagaaggt
ctgtaacttc aagaacttga tgtacaagag ctacgagttt aaaggctgtc ctgcaggggt
tgattcagtc ttcgtgtacc ccgtggctct gagctgtgag tgcaaccagg ttaactcaga
cacaacagac tggggagcta tcagcccgca gaccaccagc tgcagcatac actagagcac
tgtatcatga ccttaacaac atgtacgttg cagaatcaaa ttaagtaagg agtacaatta
gaccatttaa ggatatcaat tatttacaaa acctttagtt tttcatgcat cccacacaca
tggtaatttg gttacttgaa ttaatctgtt gtgttaattc tatagttggt actatggtaa
ctagagtact agagtataca atgctatact agttttaatt acagttaatt atagaaaagt
atgctacagt atttattaca gtttttctgt tttcaatatt tagtactaca gtatgctagt
gcattcatta acaataagct gtaaatacta taataaatac aggttaatac actttactat
agtatgcttg atcaacacta ttatttaatg tgagttacta tagtactttt caattgggat
ttgtcatttt ggatattgtg ggcttttttg gctattcata aagttttttt tatttttttt
ttatttaatt ttcagtcaaa tggaaacaag tccaccataa tacacttgtg tttcttttgt
caaacttatc aatttgtgtc tgtagatttc aattacaata catattttaa aggccaaaaa
aaaaaaaaaa aaaaaaaa

The sequence used for the design of the primers of the α subunit was:

(SEQ ID NO: 3)
gaagacactc atcacgctcc gccggaagtc gaggacaaag ccatcatgtt ttggacaaga
tacgctgaag caagcatttt cttgttgtta atgattcttc atgtcggaca actgtattca
agaaacgatg tgtctaacta tggatgtgaa gagtgcaaac tcaagatgaa cgaacgtttc
tccaaacccg gggctccggt ctatcagtgc gtgggctgct gcttttcgag agcttacccc
acacccctga ggtccaagaa aaccatgctt gtcccaaaaa acatcacatc agaagccact
tgctgtgtag caaaagaatc taaaatggtt gccacgaata tcccactata caaccacaca
gactgccact gcagcacctg ttactatcat aagtcttaaa acacactctc ttcacatttc
tcaaatgctc atttcctgtt cttaaatcac agtgactcat gaaatatgat ttttatgtag
ctttccatat ttcaactgtg gccatttcca attcgtttct aaaatggttg gcataagtat
tgtaaactgc atattctgtc actatccctt taagagcgta atatgccatc ctttactatc
attaaatcgc ttatttattt tgttgccttt actgtgacat tcttcaaatc tataaatgaa
ataaaagatt gctgaaggca aaaaaaaaaa aaaaaaaaaa aaaaaaaaaa

In each case, the gray part represents the coding sequence, and the predicted amplified region is underlined.

The primers designed were:

Upper-LHβ/EcoRI
(SEQ ID NO: 4)
5′-CAA CCG AAT TCA ACG CCT TCA AGA TGT-3′
Lower-LHβ/EcoRV
(SEQ ID NO: 5)
5′-CCG ATA TCT AGT ATG CGG GGA AAT-3′
Upper -FSHβ3
(SEQ ID NO: 6)
5′-AGG ATG CGT GTG CTT GTT CT-3′
Lower-FSHβ2/3
(SEQ ID NO: 7)
5′-TGT TGT TAA GGT CAT GAT ACA GTG C-3′
Upper-α1
(SEQ ID NO: 8)
5′-GTC GAG GAC AAA GCC ATC AT-3′
Lower-α1
(SEQ ID NO: 9)
5′-TGC CAA CCA TTT TAG AAA CGA-3′

To facilitate further cloning steps, the LHβ oligos include restriction enzyme sites for EcoRI and EcoRV in the upper oligo and in the lower oligo, respectively.

Total RNA was isolated from zebrafish heads homogenized in liquid nitrogen and extracted using TRIZOL reagent according to the manufacturer's instructions. Traces of DNA were removed by incubation with DNaseI followed by phenol/chloroform extraction and ethanol precipitation. RT-PCR was performed using the Superscript II one step RT-PCR system with platinum Taq. Reactions were performed with 100 ng of total RNA using 25 pmol of the upper and lower primers. Reverse transcription was performed at 50° C. for 30 minutes. PCR conditions were 40 cycles of denaturation at 94° C. for 20 seconds, annealing at 55° C. for LHβ and for FSHβ and 50° C. for 30 seconds and extension at 72° C. for 1 minute followed by a final extension step at 72° C. for 10 minutes. The PCR products were separated by electrophoresis in a 1% gel of agarose and stained with ethidium bromide. The FSHβ PCR product produced a faint band when observed in the agarose gel. In order to optimize, we did a PCR, using this PCR product as template. The reaction was performed with 1/20 of the PCR product reaction using 10 μM of the upper and lower primers. PCR conditions were 40 cycles of denaturation at 94° C. for 20 seconds; annealing was performed in a gradient at from 50° C. to 60° C. for 30 seconds and extension at 72° C. for 1 minute followed by a final extension step at 72° C. for 10 minutes. A sharp band was then observed. To confirm the identity of the amplified sequences, the PCR products were cloned in PCRII-TOPO vector, digested with restriction enzymes to identify the correct direction and sequenced. The analysis of the sequence revealed that we had cloned LHβ, α and FSHβ subunits of zebrafish. These constructs now allow sub-cloning the genes under a constitutive promoter.

### Cloning LHβ, FSHβ and α Under the Control of a Constitutive Promoter (CMV)

The p3XFLAG-CMV-9 expression vector is used to establish transient or stable fusion proteins. The vector encodes three adjacent FLAG epitopes upstream from the multicloning region. This results in an increased detection using anti-FLAG antibody. The promoter-regulatory region of the CMV drives transcription of flag fusion constructs. The preprotrypsin leader sequence precedes the FLAG sequence, promoting the secretion of the protein. The amino glycoside phosphotransferase gene (Neo) confers resistance to amino glycosides such as Geneticin (G418), allowing for selection of stable transfectants.

We used this vector because it has the advantages that the synthesized proteins will be secreted driven by the preprotrypsin leader. We can detect the expression of the proteins by Western blots with the anti-FLAG antibody and we can make stable cell lines selecting with geneticin.

### Cloning of LHβ

The PCR product was purified and digested with EcoRI and EcoRV, as well as the p3XFLAG-CMV-9 expression vector. The DNA fragments were ligated overnight at 4° C. The ligation mixture was then used to transform chemical-competent cells. Enzymatic digestions selected positive clones. Those clones that give a correct pattern of digestion were sequenced. At the moment, the clone sequenced showed an incorrect insertion of LHβ so I had to repeat this cloning step. The predicted amino acid sequence gives a protein of 187 amino acids with a molecular weight of 20593.8.

### Cloning of FSHβ

pCRII-TOPO FSHβ was digested with BamHI/NotI. The band that corresponds to FSHβ cDNA was purified and sub-cloned in the p3XFLAG-CMV-9. The positive clones were then digested with Not/EcoRV and the mug bean nuclease and religated to get FSHβ in frame with the preprotrypsin leader. This construct was designated as CMV-FSHβ. Alternatively, pCRII-TOPO FSHβ was digested with Xba/BamHI and the resulting band was subcloned in the p3XFLAG-CMV-9. The positive clones were then digested with EcoRV and re-ligated to get FSHβ in frame with the preprotrypsin leader. This construct was designated as CMV-FSHβ. Enzymatic digestions selected positive clones; those clones that give a correct pattern of digestion were sequenced. The sequencing of the two different colonies, which correspond to the different strategies of cloning, was correct. These constructs were used for the transformation of the ZF4 cell line. The predicted amino acid sequence gives a protein of 174/183 amino acids with a molecular weight of 19062.24/19965.21, respectively, to the two different strategies of cloning.

### Cloning of α

pCRII-TOPO a was digested with KpnI/XbaI and the resulting band was subcloned in the p3XFLAG-CMV-9. Enzymatic digestions selected positive clones. Those clones that give a correct pattern of digestion were sequenced. The analysis of the sequence revealed that we have cloned a in the correct orientation and in frame with the flag and the preprotrypsin leader sequence, this construct received the name CMV-α. The predicted amino acid sequence gives a protein of 190 amino acids with a molecular weight of 21083.24.

The plasmids CMV-FSHβ and CMV-α were purified by alkaline lysis with SDS using the QIAprep spin Miniprep kit. The purified plasmids were linearized with ScaI. The linearized products were separated by electrophoresis in a 1% gel of agarose and stained with ethidium bromide. The linearized plasmid was purified and quantified.

### Transfection of FSHβ in Zebrafish Fibroblast Cell Line (ZF4)

The ZF4 (ATCC number: CRL-2050) cells are fibroblast from 1 day-old zebrafish embryos. The frozen aliquot of ZF4 cells was removed from the liquid nitrogen and placed immediately on ice for 10 minutes. The thawed cell suspension is removed from the vial and diluted in 10 ml of complete medium (1:1 mixture of Dubelco's modified Eagle's medium and Ham's F12 containing 1.2 g/L of sodium bicarbonate, 2.5 mM L-glutamine, 15 mM HEPES and 0.5 mM sodium pyruvate, 10% of fetal bovine serum and penicillin/streptomycin) at room temperature. The supernatant is discarded by centrifugation at 1200 rpm for 8 minutes. The cells are resuspended in 8 ml of complete medium and transferred to a tissue flask (T25) and cultured at 28° C. The cells are examined under the inverted microscope to check for cell density. In cultures with a confluence of 80%, the medium is removed and washed with 3 ml of PBS to remove cellular debris and serum. Then 0.5 ml of trypsin solution (0.25%) is added and incubated at room temperature until the cells from the monolayer detach from the flask. When a single cell suspension has been obtained, 5 ml of complete medium is added to stop trypsinization. Viable and nonviable cells are counted using a Fuchs-Rosenthal hemocytometer. Cells were seeded in new flasks at a density of 100 to 150 cells/mm² (5×10⁵ in each T25 flask). The cells were incubated at 28° C. for a maximum of four days before the next passage.

Transfection was realized in zebrafish fibroblasts when they were 50-60% confluent using Fugene 6 following the manufacturer's instructions. The Fugene 6/DNA complex is prepared in a 6:1 ratio of Fugene 6: DNA in medium without serum. The culture medium is removed from the cells and replaced with serum-free medium. The Fugene 6/DNA complex is added drop-wise and mixed. The cells are incubated at 28° C. for 5 hours. Then the medium is removed and replaced with complete medium. As a control, Zf4 cells were co-transfected with the pEYFP-N1 plasmid and analyzed for positive cells under the Leica confocal at 16 and 24 hours after transfection. Different concentrations of DNA were used to establish the optimal concentration for this plasmid giving as a result that 1 μg of DNA in a surface of 21 cm² was the best concentration to obtain a transformation of approximately 30%.

### Detection of the Expression of LHβ, FSHβ and α Immunohistochemistry

Transfected cells were also analyzed by immunohistochemistry to detect the expression of the protein in the cells. The cells were fixated with p-formaldehyde 2% glutaraldehyde 0.1% in PBS for 10 minutes. To eliminate the fixative, cells are washed twice with PBS. Then they were permeabilized for 10 minutes with 0.2% of Triton X-100 in PBS. To reduce auto-fluorescence caused by the fixative NaBH4 2 mg/ml in PBS was added for 10 minutes. Blocking was done with 0.1% BSA-c, 0.02% cold water fish skin gelatin for 30 minutes. Incubation with the anti-FLAG antibody (1:250) was performed overnight at 4° C. in 0.1% BSA-c, 0.02% cold water fish skin gelatin. The antibody was removed and washed with 0.1% BSA-c, 0.02% cold water fish skin gelatin twice, 10 minutes each. The secondary antibody (anti-Rabbit Alexa 488) is added in a dilution 1:1000 in 0.1% BSA-c, 0.02% cold-water fish skin gelatin and incubated at room temperature for 60 minutes. Then is washed three times with 0.1% BSA-c, 0.02% cold water fish skin gelatin and mount in DABCO/Gelvatol. The analysis of the cells in the Leica confocal revealed expression of the protein in vesicles in the transfected cells. This localization is in agreement with the expected site for proteins that are going to be secreted.

### Western Blot

The different constructs were transfected using Fugene 6 in T25 flasks in duplicate, as control cells were not transfected with DNA, transfected with the empty vector and with the positive control CMV-BAP. The proteins were obtained at the third and fifth day after transfection from the supernatant. The supernatant was concentrated using the amicon columns following the manufacturer's instructions. FLAG fusion proteins were immunoprecipitated with Anti-FLAG M2 affinity gel, following the manufacturer's instructions.

Proteins samples were diluted 1:4 with sample buffer boiled and kept at −80° C. For setting the conditions of the Western blot only the positive (CMV-BAP) and negative (NO DNA) controls were analyzed. Samples were loaded in a 12% acrylamide gel, run for 60 minutes at 50 mAmp. The gels were blotted in nitrocellulose membranes. Membrane was blocked with milk 5% overnight at 4° C. and immunodetection was realized against anti FLAG antibody at a 1:250 dilution in milk 5% for one hour at room temperature. Immunodetection was revealed with ECL following manufacturer's instructions. The antibody recognized the expressed BAP protein showing a band of the predicted size. With these experiments, we show that transient transfected cells are producing the proteins of interest.

### Making Stable Cell Lines

Cells were transfected in 6-well chambers with linearized and purified DNA using Fugene 6. As a control, cells were transfected without DNA. After 3 or 5 days transfection, the complete medium was replaced with complete medium with G418 added. The amount of G418 to kill cells that are not expressing the construct varies from cell line to cell line. For the ZF4 cell line, the concentrations suggested by the manufacturer are between 0.8 and 1 mg/ml. Cells were treated with 0.8 and 1 mg/ml in quadruplicate; media was changed daily to wash out the dead cells. This was done for 15 days until the plates that contained the cells that did not have DNA were dead and we could not observe any cell in the plate. Then the transfected cells were incubated with the same concentration of G418 for another 5 days. When a confluent monolayer was obtained, the cells were subcultured into a T25 flask and the concentration of G418 was lowered to 0.5 mg/ml in complete media. Stable cell lines will be tested with immunohistochemistry and Western blot analysis with anti-FLAG antibody to detect the expression of the protein.

### Bioassay

In order to test if the hormones expressed by the transfected cells are active, a simple bioassay is going to be performed. Follicular cell cultures from zebrafish respond to pituitary extracts and/or human chorionic gonadotropin (hCG) by upper- or down-regulating the expression of different genes. hCG (15 IU/ml) increases the expression level of activin βA in a time-dependent manner. This effect is evident at 40 minutes of the treatment and reached a maximal level at 2 hours, longer treatment (4 hours) causes a diminishing of the effect. In contrast, activin βB is suppressed in the same conditions. When experiments using different concentrations of hCG are performed, a dose-dependent response is observed. Goldfish pituitary extract also stimulates expression of activin βA and suppresses activin βB in a dose-dependent manner. We plan to use this characteristic of the follicular cells in culture to test if the FSHβ and LHβ are active.

### In Vitro Follicular Cell Culture

### Isolation of Follicular Cells

Young zebrafish were purchased from a pet store and maintained without separation of males and females. Females were anesthetized with 0.01% tricaine methansulfonate solution for 2 minutes or until they were standing still, and decapitated before dissection. The ovaries were then removed and placed in a 10 mm culture dish with L-15 (Gibco). The follicles from five females were carefully separated with the aid of insulin needles. The separated follicles were measured with an ocular micrometer in a dissecting microscope and the healthy viotellogenic follicles around 0.45 mm were selected, pooled and cultured in T25 flask for 6 days in M199 medium supplemented with 10% fetal bovine serum at 28° C. and 5% CO₂. The medium is changed on the third day of the incubation. During the six-day incubation, follicle cells proliferated significantly, increasing the yield of cells for the experiments. Cells are washed and trypsinized at 28° C. for 15 minutes. Thereafter, the cells are washed three times with medium M199 through centrifugation at 1000 rpm for 2 minutes, and then subcultured in 24-well plates at a density of 1×10⁵ cells/ml per well for 24 hours in complete M199 before hormone treatment. The amount of cells was not enough for the experiment, so we should start with 20 females to get enough material.

### Hormone Treatment

Different concentrations of the supernatant will be used. As a positive control, hCG will be used at a 15 IU/ml, and carp pituitary extracts will also be included. With the pituitary extract condition, we can compare the amount of cells that should be used to observe an effect in the reproduction capacity of the female eel.

### In Vivo Injection

hCG was dissolved in 0.9% of NaCl solution in a concentration of 20 IU/ml. Each fish will receive 50 μl of saline as a negative control, hCG as a positive control and different concentrations of the supernatant or the purified FSHβ and/or LHβ. At 1, 2, 4, 6 and 12 hours after injections, fish are killed and ovaries removed for RNA extraction

### RNA Extraction

At the end of the hormonal treatment, total RNA was isolated from zebrafish ovaries or follicular cells, homogenized in liquid nitrogen and extracted using TRIZOL reagent according to the manufacturer's instructions. Traces of DNA were removed by incubation with DNaseI followed by phenol/chloroform extraction and ethanol precipitation. RT-PCR was performed using the Superscript II one step RT-PCR system with platinum Taq. Reactions were performed with 100 ng of total RNA using 25 pmol of the upper and lower primers. Reverse transcription was performed at 50° C. for 30 minutes. PCR conditions were 40 cycles of denaturation at 94° C. for 20 seconds, annealing at 56° C. for 30 seconds and extension at 72° C. for 1 minute followed by a final extension step at 72° C. for 10 minutes. The PCR products were separated by electrophoresis in a 1% gel of agarose and stained with ethidium bromide.

Designed primers to amplify Activin βA, Activin βB and βActin:

Upper-Activin A
(SEQ ID NO: 10)
5′-TGC TGC AAG CGA CAA TTT TA-3′
Lower-Activin A
(SEQ ID NO: 11)
5′-CAT TCG TTT CGG ACT CAA G-3′
Upper-Activin B
(SEQ ID NO: 12)
5′- CAA CTT AGA TGG ACA CGC TG-3′
Lower-Activin B
(SEQ ID NO: 13)
5′-GTG GAT GTC GAG GTC TTG TC-3′
Upper-β Actin
(SEQ ID NO: 14)
5′-CCC CTT GTT CAC AAT AAC CT-3′
Lower-β Actin
(SEQ ID NO: 15)
5′-TCT GTG GCT TTG GGA TTC A-3′

### Transplant of the Stable Cell Lines in the Eel

Stable cell lines will be transplanted to the eel intraperitoneally. Eels will be anesthetized and a small incision will be made in the belly. The correct amount of cells that produce a constant amount of hormone will be transplanted. As a negative control, cells that do not express hormones and only have the empty vector will be transplanted, and as positive control, a group of eels will be injected with pituitary extracts.

### Measurement of the Maturation Process and Egg Ripening

To quantify final maturation of female eels, there are different characteristics that change along the maturation process; those are a significant increase of the body weight, increase in the eye diameter and morphological changes during the development of the oocytes. Seven morphological stages of final oocyte maturation are observed during pituitary extract treatments. Non-transparent oocytes (stage 0) are still small and fully filled with fat droplets. Final hydration onsets development into stage 1, showing oocytes with increasing transparency and a centered nucleus. In stage 2, the oocytes are fully transparent and fat droplets are clustering and centering. In stage 3, germinal vesicle (GV) migration occurs. In stage 4, the germinal vesicle is found in the periphery and the fat droplets are located on the opposite side. In stage 5, the fat droplets decrease due to the fat fusion, giving as a result larger fat droplets. At stage 6, meiosis II is completed with disappearance of the germinal vesicle and small numbers of large fat droplets are observed. In stage 7, the oocytes have a single fat droplet. There is a change in the diameter of the oocytes during the first two stages; this is because of the process of hydration. These morphological changes in the oocyte maturation will allow us to determine the effect of the cell transplantation. After transplantation of the cells, eels will be weighed and the eye diameter will be measured regularly. During the treatment, oocyte samples will be taken in order to determine if the oocytes are responding to the treatment.

Implants with Hormone-Producing Cells

### Introduction

A test was carried out to compare the effectiveness of implantation with hormone-producing ZF4 cells with the standard technique of injection with CPE (carp pituitary extract). Sexual maturation can be obtained by repeated injection with CPE as has recently been described by Palstra et al. (2005). The repeated injections do not result in quality eggs and viable offspring, likely due to induced stress. Thus, the CPE injection protocol has to be replaced by a less stressful procedure, such as implants with hormone-producing cells, as described in this example. As the effect of CPE in eel can be observed after a few weeks by morphological, histological, and endocrinological changes, a four-week test was carried out with female silver eels. The amount of injected cells was related to the estimated weight of the pituitary of the experimental eel (ca 6 μl tissue/kg eel).

### Materials and Methods

### Hormone-Producing Cells

Three different stable cell lines were grown independently: genes for FSHβ, LHβ, and FSH/LHα were inserted in the CMV-promoter and stably transfected in ZF4 cells. According to the protocol as described above (transfected with Fugene 6 and selected with G418).

### GFP-β-Galactosidase Stable Cell Line

The pMP2838 plasmid was used to make a ZF4 stable cell line that expresses a fusion protein between the green fluorescent protein (GFP) and the β-galactosidase protein. This plasmid was described by Bakkers (2000). Briefly, the gfpN-LacZ gene of the pUAS-gfpN_LacZ plasmid was taken out and used as replacement for the gfp gene in the pEGFP-C3 plasmid. So, a green fluorescent fusion protein with β-galactosidase activity (gfpN-LacZ) was expressed under control of the CMV promoter. The pMP2838 plasmid also contains the neomycin-resistant (Neor) gene that allows the selection of positive clones with gentamicin (G418) as described above for making stable cell lines. In the same way, ZF4 stable cell lines were generated with the same protocol as described above (transfected with Fugene 6 and selected with G418).

At the day of the injection, cells were harvested and quantified to a total of 10⁸ cells for each cell line. The four cell lines were mixed to equal cell concentrations and diluted in DMEM-F12 medium without serum to a final concentration of 20 million cells in one ml.

### β-Galactosidase Tissue Staining

The tissue is briefly rinsed in PBS (phosphate buffered saline) and fixed immediately with paraformaldehyde 1%-glutaraldeyde 0.1% in PBS with MgCl₂ 2 mM, EDTA 5 mM and NP-40 0.02% for 30 minutes at room temperature. Then, it is washed twice for 5 minutes with wash solution (PBS with MgCl₂ 2 mM, EDTA 5 mM, NP-40 0.02% and Na deoxycholate 0.01%) at room temperature. Thereafter, the tissue is stained for 12 hours with stain solution (PBS with MgCl₂ 2 mM, NP-40 0.02%, Na-deoxycholate 0.01%, K₃Fe(CN)₆ 5 mM K₄Fe(CN)₆ 5 mM and X-gal 1 mg/ml) at 37° C. The stained tissue is then washed with PBS and embedded in paraffin.

### Animals and Protocol

Sixty female silver eels (900-1600 g) were caught in the wild during their seaward migration in Lake Grevelingen (Netherlands). All eels were upon arrival equipped with a microchip (Trovan) for identification, and external parameters were measured. Ten eels were immediately sacrificed as control animals. The remaining female eels were kept in a recirculation system of 3000 liters in artificial seawater (35 promille) at a temperature of 18° C. To prevent bacterial infections at the day of handling, the animals were exposed for 3 hours to the antibiotic Flumequine (50 mg/L) in a large separate tank. One group of 24 eels was injected weekly with Carp Pituitary Extract (CPE: 20-mg/kg) according to the method described before (Palstra et al. 2005), the other group was injected only once at the start of the experiment with 1 ml of a mix of four types of cells (β-gal, LHβ, FSHβ, LH/FSHα). The cells were injected as a suspension subcutaneously below the beginning of the dorsal fin and above the lateral line.

Each week, six eels from each group were sacrificed to analyze the treatment effects. These included: total weight, eye index, gonad weight, and pectoral fin length. Furthermore, tissue samples (blood, pituitary, liver, gonad) were taken for later analysis. Additionally, samples from the places of injection were obtained to test the presence of β-galactosidase-positive cells. From the same eel control samples were obtained from regions where no cells were injected.

### Results and Discussion

### Cell Implants

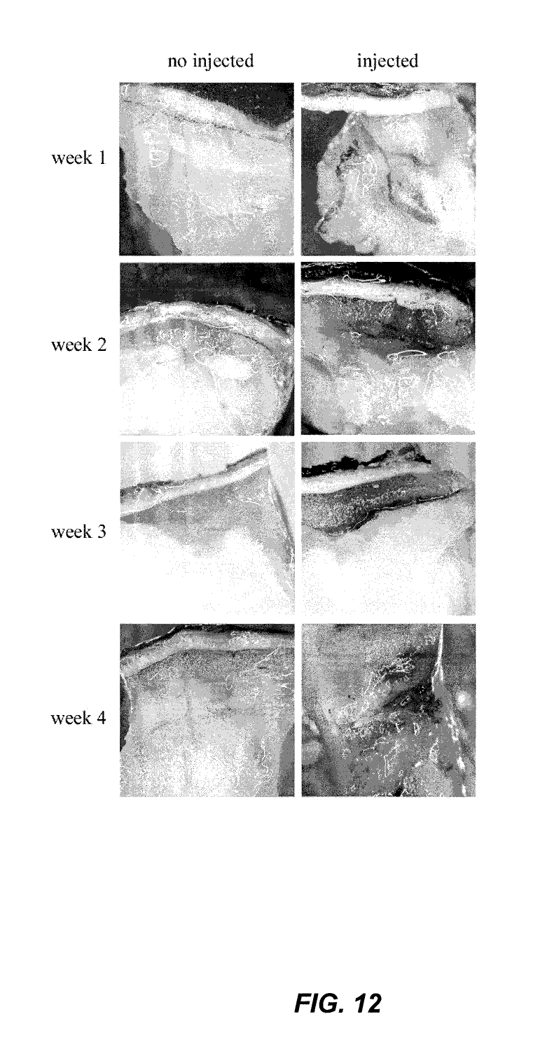

The histological analysis of the subcutaneous implants were checked for β-galactosidase staining. All samples showed the occurrence of the cells (FIG. 12). The coloring occurred initially in the fat/collagen tissue that is typical for the subcutane layers, however, also, in many cases, infiltration around the muscle layers could be observed. Comparing the samples between week 1 and week 4, there was often even an increase of the total mass of β-galactosidase-staining cells, clearly indicating that those cells are capable of infiltrating the host tissue. There was no indication for rejection, this is a remarkable observation since ZF4 cells originate from zebrafish, which is evolutionarily very distant from eel.

### Morphological Changes

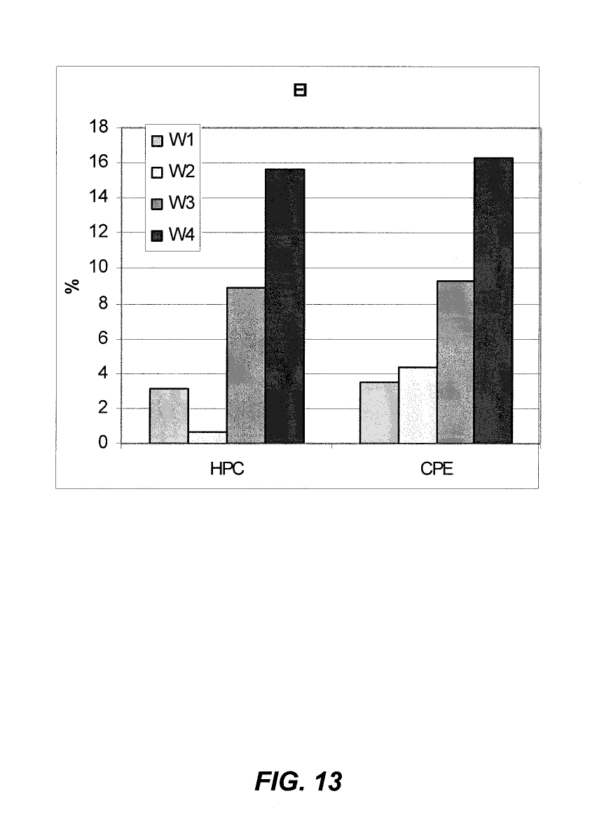

The most prominent and proximate change that indicates the onset of maturation is the increase of the eye size in silver eels (Durif et al. 2004). From FIG. 13, it is clear that the observed changes in eye index—[surface area relative to length, Pankhurst 1982]—induced by the cell implants are almost identical to those induced by CPE injection. This result clearly shows the effectiveness of cell implants. Further proof is to be obtained from a 3-4 month stimulation of silver eels, which should result in final maturation.

### CONCLUSIONS

At least two conclusions can be drawn from the above experiment: 1) zebrafish ZF4 cells (fibroblast cell line) are not rejected by silver eels over a period of four weeks and are even found to proliferate. 2) The implanted mix of cells had a similar effect on the eye index as did the standard protocol with CPE, thus showing evidence for hormonal stimulation.

Using the protocol and constructs described above, eel cell lines are generated and transplanted into eels. Further fish cells expressing both the units (α+β) are generated.

### REFERENCES

Palstra A. P., E. G. H. Cohen, P. R. W. Niemantsverdriet, V. J. T. van Ginneken, and G. E. E. J. M. van den Thillart (2005). Artificial maturation and reproduction of European silver eel: Development of oocytes during final maturation. Aquaculture, 2005.
Durif C., S. Dufour, and P. Elie (2005). The silvering process of the eel: a new classification from the yellow resident stage to the silver migrating stage. J Fish Biol. 66:1-19.
Pankhurst W. N. (1982). The relation of visual changes to the onset of sexual maturation in European eel, Anguilla anguilla L. J Fish Biol. 21:179-196.
Bakker J. (2000). Chitin oligosaccharides in Zebrafish development. Investigations at the molecular and cellular level. (Thesis University of Leiden.)

1. A method for improving the development and/or maturation of eggs and/or sperm in fish using hormone administration, said method comprising:
providing said fish with cells producing said hormone so as to improve the development and/or maturation of eggs and/or sperm in the fish.

2. The method according to claim 1, wherein said hormone comprises luteinizing hormone (LH), follicle stimulating hormone (FSH) or chorionic gonadotropin (CG) or a functional part, derivative and/or analogue thereof.

3. The method according to claim 1, wherein said cells are genetically modified to express said hormone.

4. The method according to claim 1, wherein said hormone is from the same genus as said fish.

5. The method according to claim 1, wherein said cells have been provided with one or more genes encoding said hormone.

6. The method according to claim 1, wherein said cells are a clonal population.

7. The method according to claim 1, wherein said cells have been selected for a reduced immunogenicity in said fish.

8. The method according to claim 1, wherein said fish are diadromous fish.

9. An isolated and/or recombinant fish cell that produces a fertility hormone.

10. The fish cell according to claim 9, wherein said cell is genetically modified to express said hormone.

11. The fish cell according to claim 9, wherein said cell is an eel cell.

12. A fish comprising the fish cell of claim 9.

13. The fish according to claim 12, that is a consumer fish.

14. The fish according to claim 13 that is an eel.

15. The fish according to claim 14, wherein said eel belongs to the genus Anguilla.

16. The method according to claim 2, wherein said cells are genetically modified to express said hormone.

17. The method according to claim 16, wherein said hormone is from the same genus as said fish.

18. The method according to claim 17, wherein said cells have been provided with one or more genes encoding said hormone.

19. The method according to claim 18, wherein said cells are a clonal population.

20. A fish cell genetically modified to express a hormone selected from the group consisting of luteinizing hormone (LH), follicle stimulating hormone (FSH), and chorionic gonadotropin (CG), said fish cell comprising:
one or more exogenous genes encoding said selected hormone,
wherein the fish cell is an eel cell of the genus Anguilla and the selected hormone is of the genus Anguilla.


SEQUENCE LISTING (15 sequences)

SEQ ID NO: 1 - DNA, 958 bp, Danio rerio
atatataaatctggacacgcagagacacttacaacagcctgctgagcaaccgcaacgcctgtcaagatgttattggctggaaatggtgtcttctttctcttctctttgtttttcctgctggcggctgctcagagcttggtttttccacgctgtgagctagtaaatgagacggtatcggtggaaaaagagggctgtccaaaatgcctggtgtttcagaccaccatctgcagcggccactgcgtaacaagggatcccgtttacaagagcccgttttccaccgtccaccagacagtgtgcatgtaccgggacgtccgctatgagaccattaacctgcccgactgttccgccggcgtggacccgcagatcacatacccggtggcgctgagctgcgactgcagtctgtgcaccataaacacttccgactgcaccatccagagcctgcagcccgacttctgcatgtcccagagagaggatttccccgcatactagacctcgggcaactcacgtcaacctacgcacatagtcgagctcagcattattagccctcctgtatgttttttccattaatatatatactttcaagacactagtattcagcttaaagtgacatttaaagactaaactaggttaattagggggaaaagtagagtaagtcattgtataatagtggtttgttctggagacaatccaaaactaatattgcttaagggggctaataaaattgaccttaaaatgaatttaaataatttaaaaactgcatttattctagtcgaaataaaagaaataagactttctttagaagaaaaaacattataggaaatactgcaaaaaaattcctgaatctgttcaacatcattcgggaaatcaaaggagggctaataactgtgacttcagctgtacatcaataaagaggctggttcttaaattcaaaaaaaaaaaaaaaaaaaaaaaaaaaaaa

SEQ ID NO: 2 - DNA, 1038 bp, Danio rerio
ggtctccacgaaactcccgcagatgaggatgcgtgtgcttgttctggcgctgctgttgccggtgttaatgagcgcagaatcagaatgcaggtgcagctgtcgactcaccaacatctccatcactgtggagagcgaagaatgtgggagctgcgtcacaatcgacaccacagcctgtgcaggactatgctggacaatggatcgagtttaccctagttccatggcacagcacacccagaaggtctgtaacttcaagaacttgatgtacaagagctacgagtttaaaggctgtcctgcaggggttgattcagtcttcgtgtaccccgtggctctgagctgtgagtgcaaccaggttaactcagacacaacagactggggagctatcagcccgcagaccaccagctgcagcatacactagagcactgtatcatgaccttaacaacatgtacgttgcagaatcaaattaagtaaggagtacaattagaccatttaaggatatcaattatttacaaaacctttagtttttcatgcatcccacacacatggtaatttggttacttgaattaatctgttgtgttaattctatagttggtactatggtaactagagtactagagtatacaatgctatactagttttaattacagttaattatagaaaagtatgctacagtatttattacagtttttctgttttcaatatttagtactacagtatgctagtgcattcattaacaataagctgtaaatactataataaatacaggttaatacactttactatagtatgcttgatcaacactattatttaatgtgagttactatagtacttttcaattgggatttgtcattttggatattgtgggcttttttggctattcataaagttttttttattttttttttatttaattttcagtcaaatggaaacaagtccaccataatacacttgtgtttcttttgtcaaacttatcaatttgtgtctgtagatttcaattacaatacatattttaaaggccaaaaaaaaaaaaaaaaaaaaaaa

SEQ ID NO: 3 - DNA, 710 bp, Danio rerio
gaagacactcatcacgctccgccggaagtcgaggacaaagccatcatgttttggacaagatacgctgaagcaagcattttcttgttgttaatgattcttcatgtcggacaactgtattcaagaaacgatgtgtctaactatggatgtgaagagtgcaaactcaagatgaacgaacgtttctccaaacccggggctccggtctatcagtgcgtgggctgctgcttttcgagagcttaccccacacccctgaggtccaagaaaaccatgcttgtcccaaaaaacatcacatcagaagccacttgctgtgtagcaaaagaatctaaaatggttgccacgaatatcccactatacaaccacacagactgccactgcagcacctgttactatcataagtcttaaaacacactctcttcacatttctcaaatgctcatttcctgttcttaaatcacagtgactcatgaaatatgatttttatgtagctttccatatttcaactgtggccatttccaattcgtttctaaaatggttggcataagtattgtaaactgcatattctgtcactatccctttaagagcgtaatatgccatcctttactatcattaaatcgcttatttattttgttgcctttactgtgacattcttcaaatctataaatgaaataaaagattgctgaaggcaaaaaaaaaaaaaaaaaaaaaaaaaaaaaaa

SEQ ID NO: 4 - DNA, 27 bp, Artificial
  Artificial primer
caaccgaattcaacgccttcaagatgt

SEQ ID NO: 5 - DNA, 24 bp, Artificial
  Artificial primer
ccgatatctagtatgcggggaaat

SEQ ID NO: 6 - DNA, 20 bp, Artificial
  Artificial primer
aggatgcgtgtgcttgttct

SEQ ID NO: 7 - DNA, 21 bp, Artificial
  Artificial primer
tgttgttaaggtcatgataca

SEQ ID NO: 8 - DNA, 20 bp, Artificial
  Artificial primer
gtcgaggacaaagccatcat

SEQ ID NO: 9 - DNA, 21 bp, Artificial
  Artificial primer
tgccaaccattttagaaacga

SEQ ID NO: 10 - DNA, 20 bp, Artificial
  Artificial primer
tgctgcaagcgacaatttta

SEQ ID NO: 11 - DNA, 19 bp, Artificial
  Artificial primer
cattcgtttcggactcaag

SEQ ID NO: 12 - DNA, 20 bp, Artificial
  Artificial primer
caacttagatggacacgctg

SEQ ID NO: 13 - DNA, 20 bp, Artificial
  Artificial primer
gtggatgtcgaggtcttgtc

SEQ ID NO: 14 - DNA, 20 bp, Artificial
  Artificial primer
ccccttgttcacaataacct

SEQ ID NO: 15 - DNA, 19 bp, Artificial
  Artificial primer
tctgtggctttgggattca